(19) United States
(12) Patent Application Publication
(10) Pub. No.: US 2002/0192174 A1
(43) Pub. Date: Dec. 19, 2002

(54) Perfume mixtures with increased odor intensity

(76) Inventors: Ken Kawakami, Yokohama-shi (JP); Steffen Sonnenberg, Holzminden (DE); Keith McDermott, Bound Brook, NJ (US); Leslie Smith, Princeton, NJ (US)

Correspondence Address: BAYER CORPORATION, PATENT DEPARTMENT, 100 BAYER ROAD, PITTSBURGH, PA, 15205, US

(21) Appl. No.: 10/100,337

(22) Filed: Mar. 18, 2002

(30) Foreign Application Priority Data
Mar. 23, 2001 (JP) 2001-086046

(51) Int. Cl. A61K 7/46; A61K 7/06

(52) U.S. Cl. 424/70.1; 512/1

(57) ABSTRACT

The present invention relates to perfume mixtures, their use in hair care products and hair care products containing these perfume mixtures. The odor of the perfume mixtures according to the present invention is particularly strongly perceived during or after hair-drying.

### FIELD OF THE INVENTION

The present invention relates to perfume mixtures, their use in hair care products and hair care products containing these perfume mixtures. The odor of the perfume mixtures according to the present invention is particularly strongly perceived during or after hair-drying.

### BACKGROUND OF THE INVENTION

Hair dryers are commonly used for setting or drying hair. The scent released from hair advantageously by means of a warm stream of air during the use of a hair drier produces freshness, a pleasant feeling and attractiveness. Perfume mixtures are conventionally used for hair care products with the aim of providing them with a preferred odor, of releasing a scent during the process of washing hair or of releasing a scent from dry hair.

Usually, most of the perfumes contained in hair care products, such as shampoos, hair rinses, hair conditioners or hair treatments have the tendency of escaping during the process of washing hair (such as, for example, by being washed out). In addition, some of the perfumes remaining on moist and dry hair do not have sufficient volatility on being heated by a warm stream of air. Consequently, conventional perfume mixtures do not produce the desired odorous effect on drying hair at elevated temperatures, such as, for example, with hair dryers.

Therefore, there is the need for perfume mixtures which, after applying hair care products, release a considerably enhanced scent when drying hair at elevated temperatures.

Surprisingly, it has now been found that certain perfume mixtures produce a considerably more intense and more powerful odor, i.e. an increased release of perfumes during and after the drying of wet hair at elevated temperatures.

### SUMMARY OF THE INVENTION

The present invention relates to perfume mixtures containing at least 5 perfumes having a release index of higher than or equal to 4 and an odor intensity of higher than or equal to 2.5, with the proviso that the total content of these perfumes in the overall perfume mixture is 20 to 100% by weight.

The present invention also relates to the use of these perfumes mixtures in hair care products as well as hair care products containing these perfume mixtures.

### DETAILED DESCRIPTION OF THE INVENTION

In the present invention, the release index is understood to be the quotient of the peak area (GC peak area) determined by gas chromatography of a perfume in a gas space probe above freshly dried hair at 50° C. and at 25° C. and is calculated according to the following formula:

Release index =[GC peak area at 50° C.]/[GC peak area at 25° C.]

The content of the perfume correlates with the GC peak area. Perfumes with a high release index are released particularly strongly from hair on heating it to 50° C. and thus, produce a powerful odor impression.

In the present invention, odor intensity is understood to be the odoriferous intensity of the perfume or perfume mixture released from washed hair or from a hair sample. Odor intensity was assessed by a group of experts according to the following scale: 5—very powerful, 4—powerful, 3—average, 2—poor, 1—no odor.

The perfume mixtures according to the present invention preferably contain at least 6, and more preferably at least 7 perfumes having a release index of higher than or equal to 4 and an odor intensity of higher than or equal to 2.5.

Preferred perfumes are those having a release index of higher than or equal to 5, more preferably those having a release index of higher than or equal to 6, and most preferably those having a release index of higher than or equal to 7.

Preferred perfumes are those having an odor intensity of higher than or equal to 3, more preferably those having an odor intensity of higher than or equal to 3.4, and most preferably those having an odor intensity of higher than or equal to 3.8.

The increased release of these perfumes and a correspondingly more powerful, more intense perception of the odor during and after hair-drying takes place particularly at temperatures of higher than 40° C., and preferably, at temperatures of higher than 50° C.

The perfumes contained in the perfume mixtures according to the present invention can be divided into three different odor groups A, B and C. Group A comprises perfumes having a fresh, green, citrus-like and dry-herbaceous odor. These perfumes are very frequently contained in the top note of perfume mixtures. Group B comprises perfumes of all flowery types of odor. These perfumes are frequently contained in the middle note of perfume mixtures. Group C comprises all perfumes with a woody, ambergris and musk-like odor. These perfumes are very frequently contained in the base or basic note of perfume mixtures.

Perfume mixtures according to the present invention advantageously contain at least two perfumes from group B and at least one from odor group A or odor group C.

Preferably, the perfume mixtures according to the present invention contain at least 3, and more preferably at least 4 perfumes from group B.

If the perfume mixtures only contain perfumes from group A and group B or only from group B and group C, perfume mixtures are preferred which contain at least two perfumes from each of the aforementioned groups.

Preferred perfume mixtures according to the present invention are those, which contain at least two perfumes from group B, and at least one perfume from each of groups A and C.

In order to obtain a particularly powerful odor impression, it is advantageous to select specific weight ratios between the individual odor groups A, B and C. The weight ratios are based on the sum of the perfume quantities of the respective odor group. If all three odor groups A, B and C are used in the perfume mixture according to the present invention, the weight ratio of A:B is preferably in the range from 1:2 to 1:4 and the ratio of B:C is preferably in the range from 1:1 to 4:1.

If only perfumes of odor groups A and B are used, the ratio of A:B is preferably in the range from 1:1 to 1:4.

If only perfumes of odor groups B and C are used the ratio of B:C is preferably in the range from 1:2 to 5:1.

Preferred perfumes which can be contained in the perfume mixtures according to the present invention are listed in Table 1.

TABLE 1

| Group | Preferred perfumes |
|---|---|
| A | acetaldehyde-2-phenyl-2,4-pentanediol acetal |
| A | 2,6,10-trimethyl-9-undecenal |
| A | 2-propenylcyclohexyloxyacetate |
| A | dodecanal |
| A | 1-(3-methyl-2-benzofuranyl)ethanone |
| B | gamma-undecalactone |
| B | 3-(4-tert.-butylphenyl proanal |
| B | 4-(4-methyl-3-penten-1-yl)-3-cyclohexene-1-carboxaldehyde |
| B | 2-methyl-3-(4-tert.-butylphenyl) propanal |
| B | 2-methyl-3-(4-isopropylphenyl) propanal |
| B | 3-(3-isopropylphenyl) butanal |
| B | beta-damascenone |
| B | beta-damascone |
| B | delta-damascone |
| B | 5-hexyldihydro-5-methyl-2(3H)-furanone |
| B | alpha-hexylcinnamaldehyde |
| B | alpha-irone |
| B | beta-ionone |
| B | gamma-methylionone |
| B | methyl-2-pentyl-3-oxocyclopentyl acetate |
| B | 1-(2-benzofuranyl)ethanone |
| B | heliotropin |
| C | 3-methylcyclopentadecenone |
| C | 1-(1,2,3,4,5,6,7,8-octahydro-2,3,8,8-tetramethyl-2- |
| | naphthyl)ethanone |
| C | 1-(1,2,3,4,5,6,7,8-octahydro-2,3,5,5-tetramethyl-2- |
| | naphthyl)ethanone |
| C | acetylcedrene |
| C | oxacyclohexadecen-2-one |
| C | dodecahydro-3a,6,6,9a-tetramethylnaphtho[2,1-b]furan |
| C | cyclopentadecanolide |

More preferred perfumes which can be contained in the perfume mixtures according to the present invention are the following from group A:2,6,10-trimethyl-9-undecenal, 2-propenylcyclohexyloxyacetate and 1-(3-methyl-2-benzofuranyl)ethanone; the following from group B: gamma-undecalactone, 2-methyl-3-(4-tert.-butylphenyl)propanal, 3-(3-iso-propylphenyl)butanal, 5-hexyldihydro-5-methyl-2(3H)-furanone, alpha-hexylcinnamaldehyde, alpha-irone, beta-ionone, gamma-methylionone, methyl-2-pentyl-3-oxocyclopentyl acetate, 1-(2-benzofuranyl)ethanone and heliotropin; and the following from group C:3-methylcyclopentadecenone, 1-(1 ,2,3,4,5,6,7,8-octahydro-2,3,8,8-tetramethyl-2-naphthyl)ethanone, 1-(1,2,3,4,5,6,7,8-octahydro-2,3,5,5-tetramethyl-2-naphthyl)ethanone, acetyl cedrene, oxacyclohexadecen-2-one, dodecahydro-3a,6,6,9a-tetra-methyinaphtho[2,1-b]furan and cyclopentadecanolide.

The content of the perfume mixture according to the present invention in relation to the total perfume mixture contained in the perfumed product, e.g. hair care products, is between 20 and 100% by weight. Below a content of 10% by weight in the total perfume mixture, the perfume mixture according to the present invention did not make any major contribution for the purposes of the present invention. Preferably, a content of the perfume mixture according to the present invention in the total perfume mixture of at least 25% by weight, more preferably of at least 30% by weight, and most preferably of at least 40% by weight, is preferred. In a further preferred embodiment, the content of the perfume mixture according to the present invention in the total perfume mixture is at least 50% by weight, and in a more preferred embodiment, is at least 60% by weight.

The total perfume mixture contained in the hair care product can contain additional perfumes in addition to the perfume mixtures according to the present invention.

Examples of perfumes with which the perfume mixtures according to the present invention can be advantageously combined are contained for example in K. Bauer, D. Garbe and H. Surburg, Common Fragrance and Flavor Materials, 3rd Ed., Wiley-VCH, Weinheim, 1997.

The following may be mentioned as examples:

Extracts from natural raw materials such as essential oils, concretes, absolutes, resins, resinoids, balms, tinctures, and fractions thereof or ingredients isolated therefrom; individual perfumes from the group comprising hydrocarbons; aliphatic alcohols; aliphatic aldehydes and their 1,4-dioxacycloalken-2-ones; aliphatic ketones and their oximes; aliphatic sulphur-containing compounds; aliphatic nitriles; aliphatic carboxylic acids and their esters; acyclic or cyclic terpene alcohols and their formates, acetates, propionates, isobutyrates, butyrates, isovalerates, pentanoates, hexanoates, crotonates, tiglinates or 3-methyl-2-butenoates; acyclic or cyclic terpene aldehydes and ketones; cyclic, araliphatic or cycloaliphatic alcohols; cyclic and cycloaliphatic ethers; cyclic and macrocyclic ketones; cycloaliphatic aldehydes and ketones; esters of cyclic or cycloaliphatic alcohols; aromatic hydrocarbons; esters of araliphatic alcohols and aliphatic carboxylic acids; araliphatic ethers; aromatic and araliphatic aldehydes; aromatic and araliphatic ketones; aromatic and araliphatic carboxylic acids and their esters; nitrogen-containing aromatic compounds, phenols, phenyl ethers and phenyl esters; heterocyclic compounds, lactones and macrocyclic lactones.

Hair care products according to the present invention which may be mentioned are, for example, shampoos, hair rinses, hair conditioners, hair treatments, hair sprays, hair lotions, hair foams, hair oils, hair tonics, hair creams, hair gels, pomades, permanent or semi-permanent hair colorants, hair tonics or hair restorers. Preferred hair care products are shampoos, hair rinses, hair conditioners and hair treatments.

Further ingredients which can be contained in the hair care product formulation are, for example, surfactants, silicones, cationic polymers, humectants, emolients, astringents, hair restorers, anti-itching agents, anti-inflammatory agents, alcohols, polyols, thickening agents, colorants, sunscreen filters, chelating agents, antioxidants, antimicrobial agents, preservatives, petroleum distillates and plant extracts and other conventional cosmetic additives.

The perfume mixtures according to the invention can be mixed with cosmetic additives to form a hair care product. The content in the perfume mixture according to the present invention, based on the total weight of the hair care product, is typically in the range from 0.01 to 2.0% by weight, preferably in the range from 0.1 to 1.5% by weight, and more preferably in the range from 0.2 to 1.0% by weight.

The hair care products according to the present invention can, for example, be present in the form of liquids, dispersions, milks, sprays, gels, sticks or emulsions of the oil-in-water, water-in-oil, oil-in-water-in-oil and water-in-oil-in-water type, such as for example creams or lotions.

The present invention can be explained by means of the following examples.

### EXAMPLES

### Example 1

A non-perfumed shampoo was produced by mixing the following ingredients.

TABLE 2

| Ingredient | % by weight |
|---|---|
| Sodium Laureth Sulphate | 8.0 |
| Lauramidopropyl betaine | 1.0 |
| Cocamide DEA | 3.0 |
| Glycol stearate | 2,0 |
| Polyquaterium-10 | 0.3 |
| Propylene glycol | 3.0 |
| Sodium benzoate | 0.5 |
| Water | 82.2 |
| Total | 100.0 |

### Example 2

Gas Space Analysis of a Washed Hair Sample (at 25 or 50° C.)

Shampoos were prepared by adding 0.015% by weight of a perfume according to the present invention to a shampoo formulation according to Example 1. A 3 g homogeneous hair sample was immersed in 200 ml of a 3% by weight water solution of this shampoo (40° C.). Then, this hair sample was rinsed well with 2 l of water (40° C.) and dabbed well dry with a dry towel. This hair sample was placed in a 100 ml vial, which was immediately sealed. After leaving this vial for 30 minutes at a fixed temperature (25° C. or 50° C.), the gas space of the washed hair sample in this vial was extracted by inserting for 30 minutes a solid phase microextraction fibre (PDMS/DVB 65 μm Sigma Aldrich) (gas space probe) and analyzed by GC/MS. A peak area for this perfume was recorded in a total ion chromatogram. This analysis was carried out for all of the perfumes listed in Table 3.

Conditions of the Analysis

GC/MS: Agilent Technology, HP6890 Series

Column: J&W, DB-Wax, 60 m, 0.25 mm internal diameter, 0.25 μm df

Oven temperature: 60 to 235° C. at a heating rate of 4° C./min.

Injection port: 250° C., split vent closed for 1 minute

Detector: Agilent Technology HP5973 MSD

mass range: 27-300 amu

transfer line: 240° C.

Carrier gas: Helium (30 cm/sec) (constant flow mode)

Release Index

Therefore, the quotient of the GC peak areas of the gas space probes obtained at 50° C. and 25° C. was calculated and the release index thereby determined.

### Example 3

According to the method described in Example 2, the release index was determined for each of the following: geranyl acetate, citral, dihydrimyrcenol, benzyl acetate, 4-methylene-3,5,6,6-tetramethyl-2-heptanone and linalool (Table 3).

### Example 4

Determination of the Odor Intensity

Shampoos were prepared by adding 0.015% by weight of a perfume according to the present invention to a shampoo formulation according to Example 1. A 3 g homogeneous hair sample was immersed in 200 ml of a 3% water solution of this shampoo (40° C.). Then, this hair sample was rinsed well with 2 l of water (40° C.) and dabbed well dry with a dry towel.

This hair sample was placed in a 1 l glass beaker and dried for 1 minute at about 50° C. by means of a hair drier (Matsushita Electric, EH589, 1200 W). Immediately thereafter, the glass beaker was sealed. Shortly thereafter, the glass beaker was opened and the odor intensity of the perfume released from the washed hair sample or of the released perfume mixture was assessed sensorially by a group of 5 experts.

The average value of the results determined was calculated. This evaluation was carried out for all perfumes in Table 3.

### Example 5

Determination of the Odor Intensity

Using the same procedure as in Example 4, the odor intensity was determined for dimethylbenzylcarbinyl butyrate, citral, dihydromyrcenol, benzyl acetate, 4-methylene-3,5,6,6-tetramethyl-2-heptane and linalool (Table 3).

The results from Example 2 to 5 are summarized in Table 3.

TABLE 3

| | | Release | Odor |
|---|---|---|---|
| | Perfume | index | intensity |
| Example 2 | 5-Hexyldihydro-5-methyl-2(3H)- | 11.5 | 4.0 |
| and | furanone | | |
| Example 4 | Acetyl cedrene | 11.2 | 3.2 |
| | Dodecahydro-3a,6,6,9a- | 11.1 | 4.4 |
| | tetramethyl naphtho[2,1-b]furan | | |
| | 1-(2-Benzofuranyl)ethanone | 10.3 | 3.4 |
| | 2-Propenylcyclohexyloxyacetate | 10.3 | 3.4 |
| | Oxacyclohexadecen-2-one | 10.2 | 4.0 |
| | α-Hexylcinnamaldehyde | 10.1 | 3.0 |
| | Cyclopentadecanolide | 10.0 | 4.0 |
| | 1-(1,2,3,4,5,6,7,8-Octahydro- | 10.0 | 3.4 |
| | 2,3,8,8-tetramethyl-2- | | |
| | naphthyl)ethanone and 1- | | |
| | (1,2,3,4,5,6,7,8-octahydro- | | |
| | 2,3,5,5-tetramethyl-2- | | |
| | naphthyl)ethanone | | |
| | 3-Methylcyclopentadecenone | 9.6 | 4.6 |
| | Methyl-2-pentyl-3- | 9.3 | 3.0 |
| | oxocyclopentyl-acetate | | |
| | Alpha-irone | 9.2 | 4.0 |
| | 2-Methyl-3-(4-tert.-bu- | 9.1 | 4.0 |
| | tylphenyl)propanal | | |
| | 3-(4-tert.-Butylphenyl)propanal | 7.7 | 3.2 |
| | 3-(3-Isopropylphenyl)butanal | 7.6 | 4.0 |
| | Gamma-undecalactone | 7.6 | 4.0 |
| | 4-(4-Methyl-3-penten-1-yl)-3- | 7.3 | 3.4 |
| | cyclohexen-1-carboxaldehyde | | |
| | 1-(3-Methyl-2- | 7.1 | 4.4 |
| | benzofuranyl)ethanone | | |
| | 2,6,10-Trimethyl-9-undecenal | 6.9 | 4.0 |
| | Beta-ionone | 6.8 | 4.2 |
| | Heliotropin | 6.5 | 4.0 |
| | Gamma-methylionone | 6.2 | 4.4 |
| | 2-Methyl-3-(4-isopropyl | 5.9 | 3.0 |
| | phenyl)propanal | | |
| | Acetaldehyde-2-phenyl-2,4- | 5.7 | 3.2 |
| | pentanediol acetal | | |
| | Dodecanal | 5.0 | 3.8 |
| | Beta-damascone | 4.8 | 3.4 |
| | Beta-damascenone | 4.3 | 3.8 |
| | Delta-damascone | 4.1 | 4.0 |
| Example 3 | Dimethylbenzylcarbinyl butyrate | 3.8 | 1.8 |
| and | Citral | 3.3 | 2.2 |
| Example 5 | Benzyl acetate | 2.8 | 2.0 |
| | Linalool | 1.3 | 2.2 |
| | 4-Methylene-3,5,6,6-tetramethyl- | 1.1 | 1.4 |
| | 2-heptane | | |
| | Dihydromyrcenol | 1.1 | 0.6 |

In Table 3, the release index of the perfumes preferred according to the invention shows clearly (Examples 2 and 4) that the volatilities of these perfumes from a washed hair sample surprisingly increased noticeably on heating from 25° C. to 50° C. This effect was found for release index values of 4 and higher. In addition it was found that the odor intensity of the perfumes preferred according to the invention has a surprisingly high value when the hair is dried with a hair drier at about 50° C. Upon doing so, odor intensities of 2.5 and higher were found.

By contrast, the perfumes from Example 3 and Example 5 had both a low release index of a maximum of 3.8 and a low odor intensity of a maximum of 2.2.

### Example 6 and Example 7

Two perfume mixtures were mixed which had the compositions listed in Table 4. These perfume mixtures have, in particular, a flowery scent, both as such and on the hair during and after drying.

TABLE 4

| | Perfume in % by weight | Example 6 | Example 7 |
|---|---|---|---|
| | o-Tert.-butylcyclohexyl acetate | 5.00 | 5.00 |
| | Benzyl acetate | 4.00 | 12.00 |
| | Benzyl alcohol | | 20.00 |
| | Benzyl salicylate | 4.00 | 4.00 |
| | Citronellol | 2.00 | 2.00 |
| | Citronellyl acetate | 1.00 | 1.00 |
| | Alpha-damascone | | 0.10 |
| | Dipropylene glycol | 0.90 | 0.90 |
| | Dihydromyrcenol | 4.00 | 4.00 |
| | Ethylene brassylate | 5.00 | 5.00 |
| | Geranyl acetate | 0.70 | 0.70 |
| | Cis-3-hexenol | 0.10 | 0.10 |
| | Cis-3-hexenyl acetate | 0.10 | 0.10 |
| | Limonene | 18.00 | 18.00 |
| | Linalool | 7.00 | 7.00 |
| | Linalyl acetate | 6.00 | 6.00 |
| | 4(3)-(4-Hydroxy-4-methylpentyl)- | 6.00 | 6.00 |
| | 3-cyclohexene-1-carboxaldehyde | | |
| | Phenylethyl alcohol | 8.00 | 8.00 |
| | Dimethyltetrahydrobenzaldehyde | 0.10 | 0.10 |
| | Isopropyl myristate | 0.90 | |
| * | Methyl-2-pentyl-3-oxocyclopentyl | 8.00 | |
| | acetate | | |
| * | Dodecahydro-3a,6,6,9a- | 0.10 | |
| | tetramethylnaphtho[2,1-b]furan | | |
| * | Cyclopentadecanolide | 3.00 | |
| * | Beta-damascenone | 0.10 | |
| * | Gamma-methylionone | 5.00 | |
| * | 2-Methyl-3-(4-tert.- | 8.00 | |
| | butylphenyl)propanal | | |
| * | Acetylcedrene | 3.00 | |
| | Total | 100.0 | 100.0 |

The perfume mixture from Example 7 is a traditional perfume mixture which is used as such or in a similar form in numerous shampoos. On the other hand, the perfume mixture of Example 6 also contained the perfumes (*) preferred according to the invention, the percentages by weight of the other perfumes being accordingly reduced. This did not change the type of odor of the perfume mixture to any major degree.

### Example 8

Hair Rinse

A hair rinse containing the perfume mixture according to Example 6 was produced by mixing the following ingredients.

TABLE 5

| Ingredient | % by weight |
|---|---|
| Mineral oil | 2.0 |
| Cetyl alcohol | 1.5 |
| Stearyl alcohol | 0.8 |
| Steartrimonium chloride | 0.8 |
| Glycerol | 3.0 |
| Methyl parabene | 0.1 |
| Perfume mixture from Example 6 | 0.5 |
| Water | 91.3 |
| Total | 100.0 |

### Example 9

A hair rinse containing the perfume mixture from Example 7 was prepared using the same procedure as in Example 8.

### Example 10

A 20 g homogeneous truss of hair was immersed in water (40° C.) and 1 g of the rinse prepared in Example 8 was applied to this truss. It was then rinsed well with water (40° C.) and dabbed well dry with a dry towel. This truss of hair was placed in a 10 l glass beaker, which was immediately sealed. Then the glass beaker was opened and the odor intensity of the perfume mixture released from the rinsed truss of hair was assessed by a group of 5 experts in the same way as in Example 4. (Evaluation before drying).

Then the truss of hair was dried for 1 minute with a hair drier (Matsushita Electric, EH589, 1200 W) and the glass beaker was again sealed immediately. Then the glass beaker was opened and the odor intensity of the perfume mixture released from the rinsed truss of hair was assessed by a group of 5 experts in the same way as in Example 4. (Evaluation after drying). The average values of the results were calculated.

### Example 11

The same procedure as in Example 10 was used for the hair rinse from Example 9.

The results of Examples 10 and 11 are summarized in Table 6.

TABLE 6

| | Odor intensity before drying | Odor intensity after drying |
|---|---|---|
| Example 10 | 1.0 | 4.2 |
| Example 11 | 1.2 | 1.8 |

Table 6 shows that the perfume mixture according to the present invention from Example 10, which contains perfumes preferred according to the invention, has a comparable odor intensity before drying to that of the perfume mixture of Example 11.

By contrast, the group of experts detected a considerably increased odor intensity for Example 10 according to the invention when the hair was dried with a hair drier and after drying. The perfume mixture from Example 10 was considerably preferred by one user group.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Perfume mixtures comprising at least 5 perfumes with a release index of greater than or equal to 4 and an odor intensity of greater than or equal to 2.5, wherein the total content of said perfumes in the total perfume mixture is 20 to 100% by weight, wherein the release index is defined as the quotient of the peak area determined by gas chromatography of a perfume in a gas space probe above freshly dried hair at 50° C. and at 25° C. and the odor intensity is defined as the odoriferous intensity of the perfume released from washed hair on a scale of 1 (no odor) to 5 (very powerful).

2. Perfume mixtures according to claim 1, wherein said mixtures comprise at least 6 perfumes with a release index of greater than or equal to 4 and an odor intensity of greater than or equal to 2.5.

3. Perfume mixtures according to claim 1, wherein the total amount of perfumes having a release index of greater than or equal to 4 and an odor intensity of greater than or equal to 2.5 is at least 25% by weight of the total perfume mixture.

4. Hair care products comprising perfume mixtures which comprise at least 5 perfumes with a release index of greater than or equal to 4 and an odor intensity of greater than or equal to 2.5, wherein the total content of said perfumes in the total perfume mixture is 20 to 100% by weight, wherein the release index is defined as the quotient of the peak area determined by gas chromatography of a perfume in a gas space probe above freshly dried hair at 50° C. and at 25° C. and the odor intensity is defined as the odoriferous intensity of the perfume released from washed hair on a scale of 1 (no odor) to 5 (very powerful).

5. Hair care products according to claim 4, wherein said mixtures comprise at least 6 perfumes with a release index of greater than or equal to 4 and an odor intensity of greater than or equal to 2.5.

6. Hair care products according to claim 4, wherein the total amount of perfumes having a release index of greater than or equal to 4 and an odor intensity of greater than or equal to 2.5 is at least 25% by weight of the total perfume mixture.
